(19) United States
(12) Patent Application Publication
(10) Pub. No.: US 2019/0318812 A1
(43) Pub. Date: Oct. 17, 2019

(54) RECORDING DEVICE, ORDER ISSUING DEVICE AND ANALYSIS PROCESSING SYSTEM

(71) Applicant: Konica Minolta, Inc., Tokyo (JP)

(72) Inventor: Yuuichi NISHIJIMA, Tokyo (JP)

(21) Appl. No.: 16/380,248

(22) Filed: Apr. 10, 2019

(30) Foreign Application Priority Data
Apr. 12, 2018 (JP) 2018-076492

(51) Int. Cl. G16H 10/40

(52) U.S. Cl. CPC G16H 10/40

(57) ABSTRACT

A recording device which records information regarding a test that is performed based on test order information including imaging, the recording device including a hardware processor which records a test time of each test from start of the test to end of the test, an imaging time of each imaging included in each test from start of the imaging to end of the imaging, and information regarding an event which occurs in each test and deviates from an operation determined in advance.

### CROSS REFERENCE TO RELATED APPLICATION

The present application claims priority under 35 U.S.C. § 119 to Japanese Application No. 2018-076492, filed Apr. 12, 2018, the entire content of which is incorporated herein by reference.

### BACKGROUND

### Technological Field

The present invention relates to a recording device, an order issuing device and an analysis processing system.

### Description of the Related Art

In a medical field, there has been conventionally suggested a technique of obtaining log data of operation contents, operation time and the like in a test device which performs imaging of medical images, and using the obtained data to improve efficiency of imaging.

For example, Patent document 1 (Japanese Patent Application Laid Open Publication No. 2005-293301) describes a technique of storing, in a database, information regarding a movement of a person detected by a sensor in a medical facility and performance information, and creating an analysis report of work in the medical facility on the basis of the information regarding the movement of the person and the performance information stored in the database.

However, the technique described in Patent document 1 does not consider the occurrence of events (referred to as unexpected events) deviating from operations which were determined in advance. Thus, it is difficult for a manager to specify the cause decreasing the performance. Especially in a large-scale medical facility, the numbers of imaging and the test devices are large-scale and many test technicians are arranged. Thus, it is difficult for the manager to specify the cause which deteriorates the performance without grasping the situation in which the unexpected event occurs.

### SUMMARY

An object of the present invention is to provide information on the occurrence situations of unexpected events in tests.

To achieve at least one of the abovementioned objects, according to an aspect of the present invention, a recording device reflecting one aspect of the present invention records information regarding a test that is performed based on test order information including imaging, the recording device including a hardware processor which records a test time of each test from start of the test to end of the test, an imaging time of each imaging included in each test from start of the imaging to end of the imaging, and information regarding an event which occurs in each test and deviates from an operation determined in advance.

According to an aspect of the present invention, an analysis processing system reflecting one aspect of the present invention includes: the recording device according to claim 1; and an analysis processing device which analyzes data recorded by the recording device.

According to an aspect of the present invention, an order issuing device reflecting one aspect of the present invention issues test order information, the order issuing device including a hardware processor which records a test time and information regarding an event, the test time being a test time from start of a test to end of the test based on each piece of test order information that is issued, and the event deviating from an operation determined in advance.

According to an aspect of the present invention, an analysis processing system reflecting one aspect of the present invention includes: the order issuing device according to claim 6; and an analysis processing device which analyzes data recorded by the order issuing device.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a view showing an entire configuration of an analysis processing system in an embodiment of the present invention;

FIG. 2 is a block diagram showing a functional configuration of a console in FIG. 1;

FIG. 3 is a view showing screen shift at the time of normal test;

FIG. 4 is a view showing an example of a test list screen;

FIG. 5 is a view showing an example of a test screen;

FIG. 6 is a view showing screen shift at the time of a test in which an unexpected event occurred;

FIG. 7 is a view showing an analysis result report regarding imaging time for chest upright; and

FIG. 8 is a view showing an analysis result report regarding the number of imaging errors for each imaging site.

### DETAILED DESCRIPTION OF THE EMBODIMENTS

Hereinafter, one or more embodiments of the present invention will be described with reference to the drawings. However, the scope of the invention is not limited to the disclosed embodiments.

[Configuration of Analysis processing system 100]

First, the configuration of the embodiment will be described.

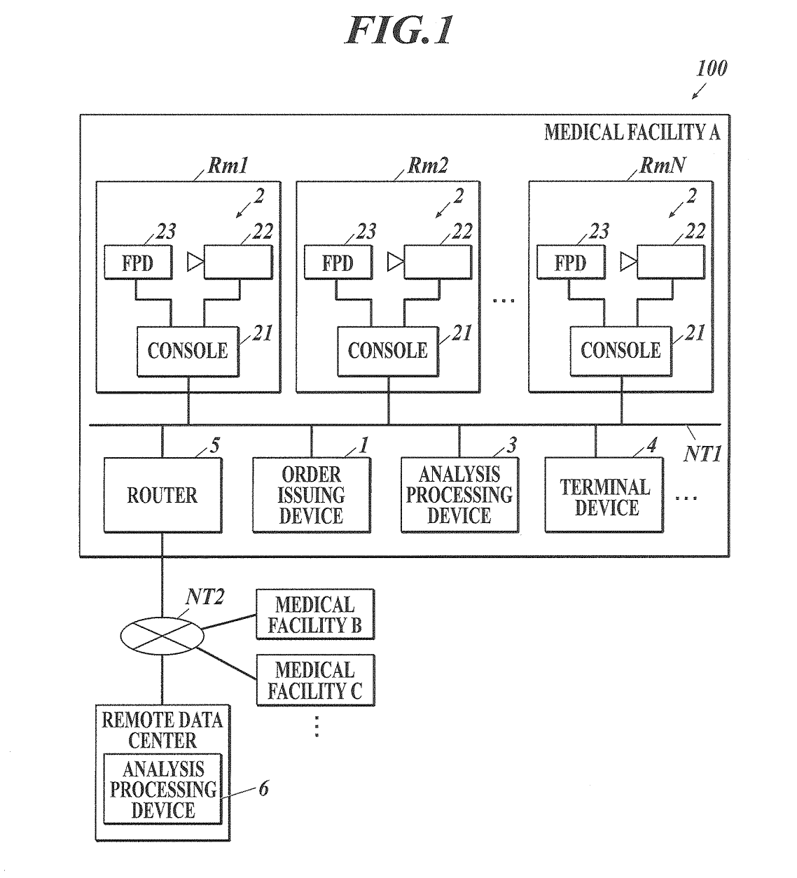

FIG. 1 is a view showing the entire configuration example of an analysis processing system 100 in the embodiment.

As shown in FIG. 1, the analysis processing system 100 includes a facility system which is set in a medical facility A, a facility system which is set in a medical facility B, a facility system which is set in a medical facility C, and an analysis processing device 6 which is set in a remote data center.

The facility system of the medical facility A is configured by connecting an order issuing device 1, a test device 2, an analysis processing device 3, and a terminal device 4 so as to be able to perform data transmission/reception with each other via a facility network NT1 such as LAN (Local Area Network). At least the analysis processing device 3 and the terminal device 4 among the devices forming the facility system can perform data transmission/reception with the analysis processing device 6 of the remote data center via an internet NT2 by a router 5.

The facility systems of the medical facilities B and C also have a same configuration as that of the facility system of the medical facility A (not shown in the drawings).

The number of medical facilities forming the analysis processing system 100 and the number of devices of the facility system in each of the medical facilities are not especially limited.

Hereinafter, each device forming the facility system in each of the medical facilities A to C and the analysis processing device 6 will be described.

The order issuing device 1 is a device for issuing test order information on the basis of a test order which is requested from a diagnosis and treatment department, and transmitting the issued test order information to the console 21 of the test device 2. The order issuing device 1 is configured by including a computer which includes: a controller formed by including a server (CPU (Central Processing Unit)), a RAM (Random Access Memory), an RTC (Real Time Clock) and the like; a storage such as a hard disk; a communicator; and the like, and an input terminal. Generally, the order issuing device 1 refers to RIS (Radiology Information System). The test order information includes, for example, patient information (patient ID, patient name), patient attribute information (sex, birth date, age and the like), test information (test ID, test date, imaging ID of each imaging in the test, imaging site (including imaging direction and posture), modality type and the like).

The test device 2 is a device (modality) which executes a test in accordance with the test order information from the order issuing device 1. The embodiment is described by taking, as an example, a case where the test device 2 is a digital X-ray imaging device. However, the test device 2 may be other modalities such as a CT (Computed Tomography), MRI (Magnetic Resonance Imaging) and an ultrasound device.

A single test device 2 is provided to each of imaging rooms Rm1 to RmN (N is a positive integer). The test device 2 is configured by including a console 21, a radiation generator 22, and an FPD (Flat Panel Detector) 23.

The console 21 performs a test by controlling the radiation generator 22 and the FPD 23 in accordance with the test order information transmitted from the order issuing device 1. The console 21 as a recording device records information regarding the test which was performed on the basis of the test order information. For example, the console 21 records a test ID, test date and time and a test time of each test, a technician ID of the test technician, imaging date and time and an imaging time of each imaging in the test, a panel ID of the used FPD 23, and an event ID, the number of occurrence and a working time of the unexpected event which occurred in the test, and the like. The console 21 saves the recorded data (referred to as recording data) for a certain period and transmits the data to the analysis processing device 3.

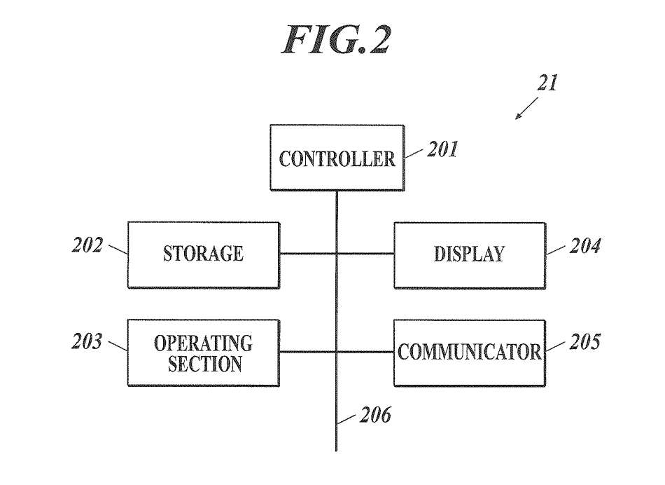

As shown in FIG. 2, the console 21 is configured by including a controller 201, a storage 202, an operating section 203, a display 204, and a communicator 205, and these components are connected to each other via a bus 206.

The controller 201 is configured by including a CPU, a RAM, an RTC and the like. In response to the operations of the operating section 203, the CPU of the controller 201 reads out a system program and various types of processing programs stored in the storage 202 and loads them into the RAM, and intensively controls the operations of components in the console 21, the radiation emitting operation of the radiation generator 22 and the reading operation of the FPD 23 in accordance with the loaded programs.

The storage 202 is configured by including a non-volatile semiconductor memory, a hard disk and the like. The storage 202 stores various programs executed by the controller 201, parameters necessary for executing processing by the programs and data such as processing results. The various programs are stored in a form of readable program code, and the controller 201 sequentially executes the operations according to the program code.

The storage 202 stores the radiation emitting condition and the image reading condition corresponding to the imaging site.

The storage 202 stores information on the movement (operation) which is a trigger starting the event and the movement (operation) which is a trigger ending the event, so as to be associated with the event ID for identifying the unexpected event in the test device 2.

The storage 202 stores the imaging room ID for identifying the imaging room in which the device is arranged, and user information (technician ID, password, and name of the test technician for identifying the test technician who is the user).

The storage 202 includes a database for storing the test order information which is transmitted from the order issuing device 1, and the above mentioned recording data. The database has, for each test, regions for storing “test ID”, “test order information”, “technician ID”, “test date and time”, “test time”, and “ imaging ID”, “panel ID”, “imaging date and time”, and “imaging time” of each imaging in the test, “event ID”, “number of occurrence”, and “working time”, for example. The “event ID”, “occurrence date and time”, “number of occurrence” and “working time” are respectively the event ID, the occurrence date and time, the number of occurrence and the working time (unexpected working time) of the unexpected event which occurred during the test.

The operating section 203 is configured by including a keyboard which includes a cursor key, numeric input keys, various function keys and the like, and a pointing device such as a mouse. The operating section 203 outputs the instruction signal, which was input by a key operation to the key board and a mouse operation, to the controller 201. The operating section 203 may include a touch panel on the display screen of the display 204. In this case, the operating section 203 outputs the instruction signal input via the touch panel to the controller 201.

The display 204 is configured by including a monitor such as an LCD (Liquid Crystal Display) and a CRT (Cathode Ray Tube), and displays the input instruction from the operating section 203, data and the like in accordance with the instruction of the display signal input from the controller 201.

The communicator 205 includes a LAN adapter, a modem, a TA (Terminal Adapter) or the like, to control data transmission/reception with the order issuing device 1, the analysis processing device 3 and the like connected to the communication network NT1.

The radiation generator 22 is arranged at a position facing the FPD 23 across the subject, and emits radiation (X ray) to the subject in accordance with control by the console 21.

The FPD 23 is provided so as to face the radiation generator 22 across the subject. For example, the FPD 23 has a glass substrate or the like. At a predetermined position on the substrate, the FPD 23 has, in a matrix, a plurality of detection elements (pixels) which detects, according to the intensity, radiation which was emitted from the radiation generator 22 and transmitted through at least the subject, and which converts the detected radiation into electrical signals and accumulates them. Each of the pixels includes a switching section such as a TFT (Thin Film Transistor), for example. The FPD 23 controls the switching section of each pixel on the basis of the image reading condition which was input from the console 21, performs switching of reading of the electrical signal accumulated in each pixel, reads the electrical signal accumulated in each pixel, and thereby obtains the captured image. The FPD 23 outputs the obtained captured image to the console 21. Each of the imaging rooms Rm1 to RmN is provided with an upright imaging table, a recumbent imaging table or the like, and the FPD 23 is used by being mounted on the imaging table at the time of imaging.

Similarly to the console 21, the analysis processing device 3 is configured by including a controller, a storage, an operating section, a display, and a communicator. The analysis processing device 3 collects recording data from the order issuing device 1 and the console 21 of each imaging room in the facility to temporarily store the recording data for a certain time, analyzes the collected recording data, and outputs (transmits) the analysis result as a report to the terminal device 4 of the facility manager and the like.

Similarly to the console 21, the terminal device 4 is configured by including a controller, a storage, an operating section, a display, and a communicator. The terminal device 4 includes a reading/interpreting terminal for a doctor to display the captured image and read and interpret the image, a manager terminal for the facility manager to view the report output from the analysis processing device 3, and the like.

Similarly to the console 21, the analysis processing device 6 is configured by including a controller, a storage, an operating section, a display, and a communicator. The analysis processing device 6 stores recording data which was transmitted from the medical facilities A to C in the database, analyzes the recording data and outputs the analysis result as a report. The analysis processing device 6 can perform analysis integrating the pieces of recording data of the tests performed at a plurality of medical facilities.

[Operation of Analysis processing system 100]

Next, the operation of the analysis processing system 100 will be described.

### (Operation of Order Issuing Device 1)

First, the operation of the order issuing device 1 will be described. The operation of the order issuing device 1 is executed by the controller (CPU) of the server included in the order issuing device 1.

When a test order is input from the input terminal, the order issuing device 1 issues test order information on the basis of the input test order, and stores the issued test order information in the storage of the server so as to be associated with the status of “standby for test”. When the test order information is selected from the test standby list (list of test order information in the status of “standby for test”) by the input terminal and the test device 2 to be used is specified, the order issuing device 1 transmits the selected test order information to the console 21 of the specified test device 2 to request starting the test, changes the status of the selected test order information from “standby for test” to “under test”, and obtains the current date and time as the test date and time (test start date and time) and starts the measurement of the test time. When the test end notice is received together with the actual result from the test device 2, the order issuing device 1 changes the status of the test order information to “test end”, and ends the measurement of the test time. The order issuing device 1 records the test date and time and the test time so as to be associated with the test order information stored in the storage.

In the order issuing device 1, reissuance, change of order contents, and change of patient information/attribute information can be performed for the test order information which is on standby for test. These operations are unexpected events which are not included in the normal operation flow of order issuance. The order issuing device 1 measures, as the working time of the unexpected event, the time during which the reissuance work of the test order information was performed on the reissuance screen of the test order information, and the time during which the change of order contents or the change work of the patient information/attribute information was performed on the change screen of the test order information. The order issuing device 1 stores (records) each time in the storage of the server so as to be associated with the event ID, the occurrence date and time (date and time at which each screen was opened) and the operator ID of the operator who performed the unexpected event.

The order issuing device 1 associates, for each predetermine period (for example, for each single day), the test date and time, and test time of each test, event ID of the unexpected event, number of occurrence, occurrence date and time, working time and the operator ID stored in the storage, and transmits them as recording data to the analysis processing device 3.

### (Operation of Test Device 2)

Next, the operation of the test device 2 will be described. First, the operation flow of normal test in which the unexpected event does not occur will be described.

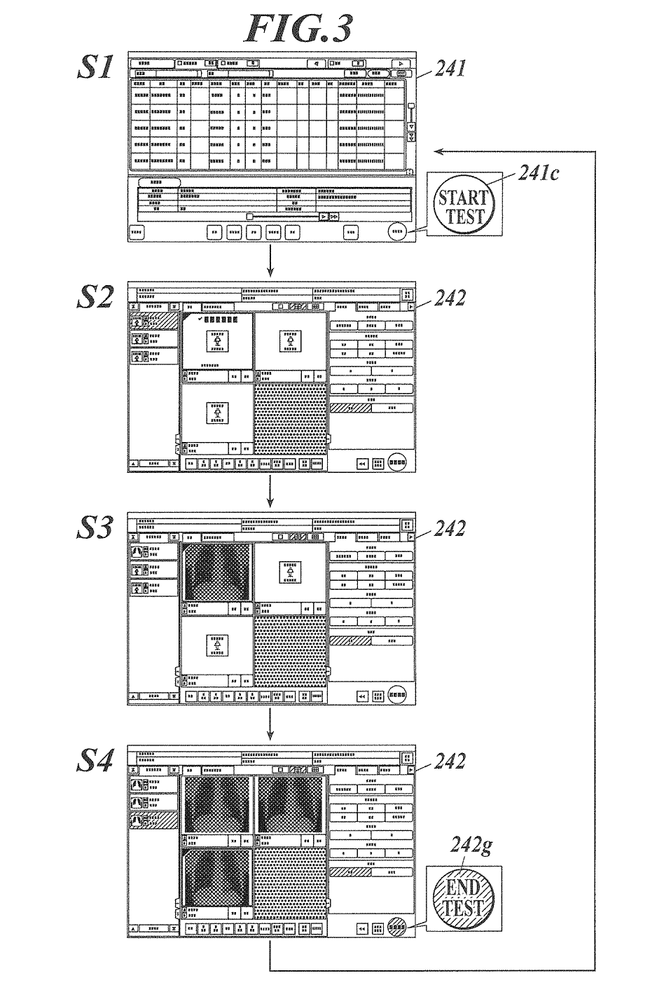

FIG. 3 is a view showing the screen shift of the console 21 in the normal test. Hereinafter, the operation flow of normal test of the test device 2 will be described by referring to the screen shift of FIG. 3.

When test order information is received from the order issuing device 1, the controller 201 of the console 21 displays the test order information on the test list screen 241 displayed as an initial screen by the display 204 (step S1).

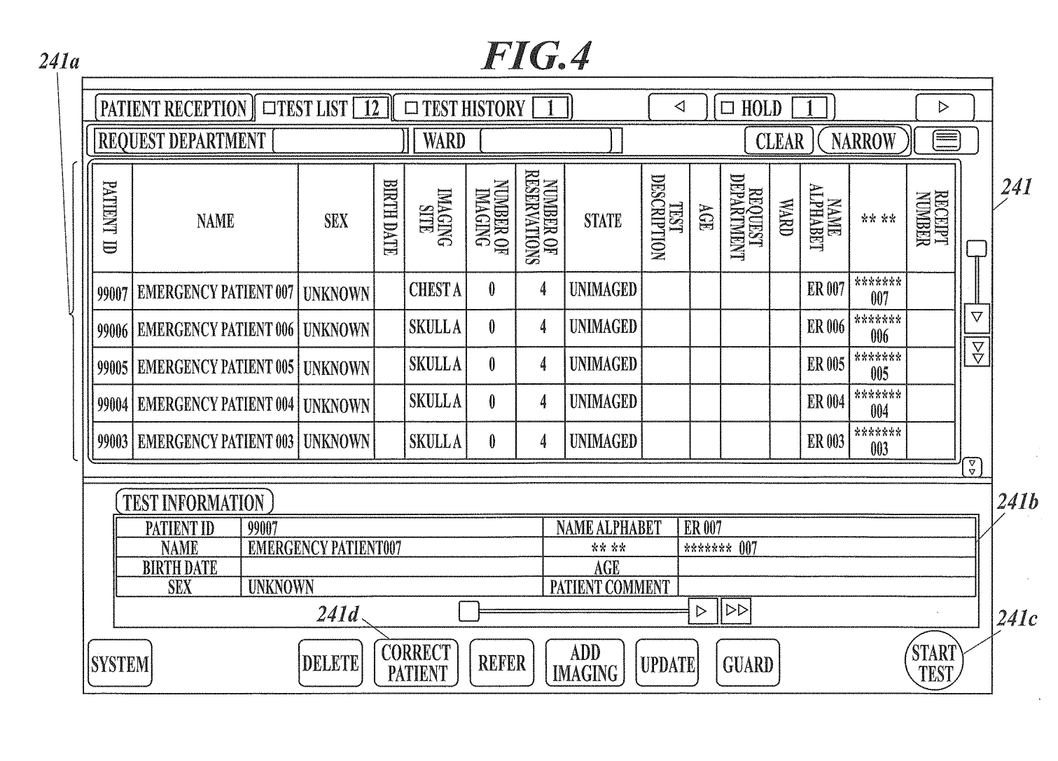

FIG. 4 is a view showing an enlarged view of the test list screen 241. As shown in FIG. 4, the test list screen 241 includes a test list display field 241a in which a list of test order information is displayed, a test information display field 241b in which the test order information selected by the operating section 203 is displayed, a test start button 241c for instructing start of the test, a patient correction button 241d for instructing correction of the patient information and the attribute information and the like.

When the test order information is selected from the test list screen 241 by the operating section 203, and the test start button 241c is pressed, the controller 201 obtains the date and time at which the test start button 241c was pressed as the test date and time, stores the test date and time in the RAM so as to be associated with the test ID, technician ID (logging in technician ID) and the imaging room ID, causes the display 204 to display the test screen 242, and starts the measurement of test time (step S2).

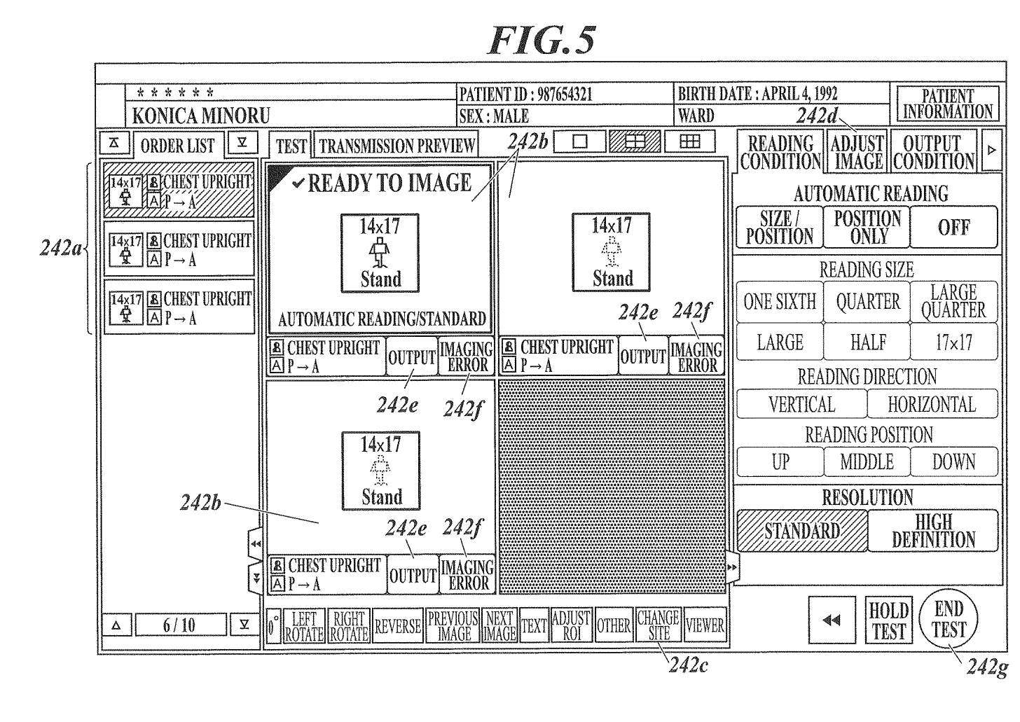

FIG. 5 is an enlarged view of the test screen 242. As shown in FIG. 5, the test screen 242 includes imaging selection buttons 242a for selecting imaging of the imaging target (processing target) from among pieces of imaging included in the test order information, image display field 242b for displaying the captured image obtained by the imaging, a site change button 242c for instructing change of the imaging site of the selected imaging, an image adjustment tab 242d for instructing image adjustment of the captured image, an output button 242e for instructing output of the captured image to the external device, an imaging error button 242f for instructing treating the captured image as an imaging error, a test end button 242g for instructing end of the test, and the like. The output button 242e and the imaging error button 242f are invalid until the captured image is received.

When an imaging selection button 242a is pressed and the imaging is selected on the test screen 242, the controller 201 obtains the current date and time as imaging date and time, and starts measurement of the imaging time. The controller 201 obtains the panel ID from the FPD 23 mounted on the imaging table (upright or recumbent) used in the selected imaging, and stores the panel ID in the RAM so as to be associated with the imaging ID, the imaging date and time and the imaging site of the selected imaging. The controller 201 respectively transmits the radiation emitting condition and the image reading condition corresponding to the imaging site to the radiation generator 22 and the FPD 23 to instruct them to start the imaging. The controller 201 is then on standby for transmission of the captured image from the FPD 23.

After selection of the imaging on the test screen 242, the test technician performs positioning of the patient, adjustment of the position and direction of the radiation generator 22, guidance of posture to the patient, and the like. When the imaging gets ready, the test technician presses the exposure button of the radiation generator 22 and performs radiation imaging. The FPD 23 accumulates and reads the electric charges corresponding to the radiation transmitted through the subject (imaging site of the patient), and transmits the reading result as the captured image to the console 21.

When the communicator 205 receives the captured image transmitted from the FPD 23, the controller 201 ends the measurement of the imaging time and stores the imaging time in the RAM so as to be associated with the imaging ID. The controller 201 then causes the display 204 to display the received captured image in the image display field 242b (step S3).

When all the pieces of imaging in the test end, the captured images are output to the terminal device 4 or the like according to the operation of the output button 242e of the operating section 203, and the test end button 242g is pressed (step S4), the controller 201 ends the measurement of the test time and notifies the end of test to the order issuing device 1. The controller 201 records the recording data (test ID, test date and time, technician ID, imaging room ID, test time, and panel ID, imaging date and time, imaging site and imaging time for each imaging) of the ended test, which is stored in the RAM, in the database of the storage 202, and causes the communicator 205 to transmit the data to the analysis processing device 3. The controller 201 stores the captured images obtained by the test in the storage 202 so as to be associated with the test order information. The controller 201 deletes the information regarding the ended test stored in the RAM, and causes the display 204 to display the test list screen 241.

Next, description will be made for the operation flow of the test in a case where unexpected events occur in the test device 2.

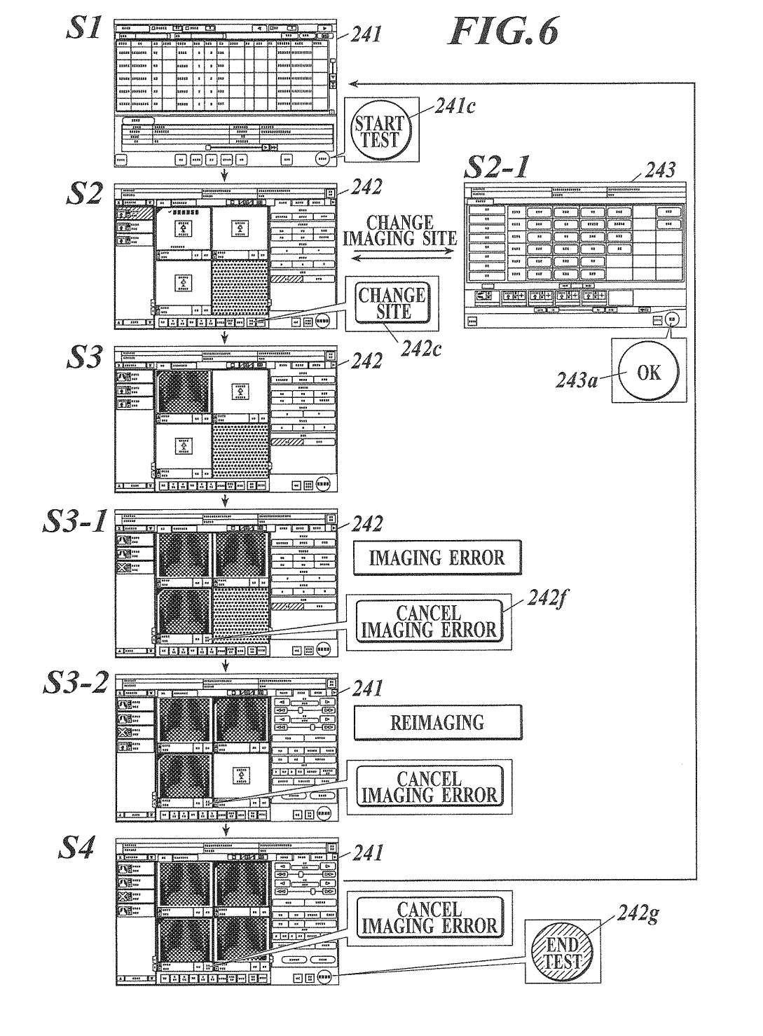

FIG. 6 is a view showing screen shift of the console 21 in the test in which change of imaging site, imaging error and re-imaging occurred as unexpected events. Hereinafter, description will be made for an example of the operation flow of the test in which unexpected events occurred with reference to FIG. 6.

When the test order information is received from the order issuing device 1, the controller 201 of the console 21 displays test order information on the test list screen 241 of the display 204 (step S1).

When the test order information is selected from the test list screen 241 by the operating section 203 and the test start button 241c is pressed, the controller 201 obtains, as the test date and time, the date and time at which the test start button 241c was pressed, stores the obtained date and time in the RAM so as to be associated with the test ID, technician ID and imaging room ID, causes the display 204 to display the test screen 242, and starts the measurement of the test time (step S2).

Here, the test technician selects one imaging selection button 242a on the test screen 242, presses the site change button 242c, and thereby can change the imaging site in the selected imaging.

When the site change button 242c is pressed in a state in which the one imaging selection button 242a is selected on the test screen 242, the controller 201 causes the display 204 to display the site change screen 243 (step S2-1).

On the site change screen 243, when the change contents of the imaging site of the selected imaging is input and the OK button 243a is pressed by the operating section 203, the controller 201 changes the imaging site of the test order information of the selected imaging on the basis of the input change contents, and returns the display of the display 204 to the test screen 242.

The operation from pressing of the site change button 242c to displaying of the site change screen 243, pressing of the OK button 243a and returning to the test screen 242 again is the unexpected event which is not included in the operation flow of the normal test mentioned above. Thus, when the imaging selection button 242a is selected and the site change button 242c is pressed, the controller 201 refers to the storage 202, obtains the event ID of the unexpected event triggered by this operation as a start trigger, and obtains the current date and time as the occurrence date and time of the event. The controller 201 measures the time from pressing of the site change button 242c until the test screen 242 is displayed again as the working time of the unexpected event, and stores (records) the event ID, the occurrence date and time and the working time in the RAM so as to be associated with each other. In a case where the site change is performed on the test screen 242 a plurality of times, the measurement and the recording are performed assuming that an unexpected event occurred for each site change.

When the imaging selection button 242a is pressed and imaging is selected on the test screen 242, the controller 201 obtains the current date and time as the imaging date and time and starts the measurement of the imaging time. The controller 201 obtains the panel ID from the FPD 23 mounted on the imaging table (upright or recumbent) used in the selected imaging, and stores the panel ID in the RAM so as to be associated with the imaging ID, imaging date and time and imaging site of the selected imaging. The controller 201 respectively transmits the radiation emitting condition and the image reading condition corresponding to the imaging site to the radiation generator 22 and the FPD 23 to instruct them to start the imaging. The controller 201 is then on standby for transmission of the captured image from the FPD 23.

When the imaging selection button 242a is pressed, the controller 201 starts the measurement of the imaging time. In a case where an unexpected event occurred, the controller 201 starts time measurement of the unexpected event while maintaining the measurement of the imaging time.

When the communicator 205 receives the captured image transmitted from the FPD 23, the controller 201 ends the measurement of the imaging time and stores the imaging time in the RAM so as to be associated with the imaging ID. The controller 201 then causes the display 204 to display the received captured image in the image display field 242b (step S3).

The test technician repeats imaging by using the test screen 242 until all the pieces of imaging in the test end.

In a case where the test technician determined that the received captured image is an imaging error due to imaging defect such as lack of imaging site and generation of body movement, for example, the test technician can instruct treating the captured image as an imaging error by pressing the imaging error button 242f corresponding to the captured image in the image display field 242b on the test screen 242.

On the test screen 242, when the imaging error button 242f is pressed, the controller 201 causes the display 204 to display a pop-up screen (not shown in the drawings) displaying a message of “imaging error?”, an OK button and a cancel button. When the OK button is pressed by the operating section 203, the controller 201 closes the pop-up screen to write that the image is an imaging error in the header of the captured image, displays “x” for the imaging selection button 242a corresponding to the captured image, and treats the captured image as an imaging error. The controller 201 changes the imaging error button 242f corresponding to the captured image to an imaging error cancel button 242f (step S3-1).

The operation from pressing of the imaging error button 242f to pressing of the OK button, changing in the imaging selection button 242a and displaying of the imaging error cancel button 242f is the unexpected event which is not included in the operation flow of the normal test mentioned above. Thus, when the imaging error button 242f is pressed, the controller 201 refers to the storage 202, obtains the event ID treating the pressing of the imaging error button 242f as a start trigger, and obtains the current date and time as the occurrence date and time. The controller 201 measures the time from pressing of the imaging error button 242f to changing of display of the imaging selection button 242a and the imaging error button 242f as a working time of the unexpected event, and stores the event ID, the occurrence date and time and the working time in the RAM so as to be associated with each other.

When the OK button is pressed on the pop-up screen of “imaging error?”, the controller 201 then causes the display 204 to display a pop-up screen (not shown in the drawings) displaying a message of “imaging again?”, an OK button and a cancel button. When the OK button is pressed by the operating section 203, the controller 201 displays an imaging selection button 242a of re-imaging immediately below the imaging selection button 242a of the imaging which was determined to be an imaging error, causes the imaging selection button 2442a of re-imaging to be in a selected state, and is on standby (step S3-2). When the communicator 205 receives the captured image, the controller 201 causes the display 204 to display the received captured image in the image display field 242b (step S4).

The operation from instruction of re-imaging to reception of the captured image of re-imaging is the unexpected event which is not included in the operation flow of the normal test mentioned above. Thus, when the OK button instructing re-imaging is pressed on the pop-up screen, the controller 201 refers to the storage 202, obtains the event ID treating the pressing of the OK button which instructs the re-imaging as a start trigger, and obtains the current date and time as the occurrence date and time of the event. The controller 201 measures the time from pressing of the OK button to reception of the captured image of the re-imaging, and stores the event ID, the occurrence date and time and the working time in the RAM so as to be associated with each other.

When all the pieces of imaging in the test end, the captured images are output to the terminal device 4 or the like according to the operation of the output button 242e of the operating section 203, and the test end button 242g is pressed, the controller 201 ends the measurement of the test time and notifies ending of the test to the order issuing device 1. The controller 201 records the recording data (test ID, test date and time, technician ID, imaging room ID, test time, panel ID, imaging date and time, imaging site, and imaging time for each imaging, event ID, occurrence date and time, and working time for each unexpected event which occurred in the test, the number of occurrence of the unexpected events, and the like) of the ended test, which is stored in the RAM, in the database of the storage 202 so as to be associated with each other, and causes the communicator 205 to transmit the data to the analysis processing device 3. The controller 201 stores the captured images obtained by the test in the storage 202 so as to be associated with the test order information. The controller 201 deletes information regarding the ended test stored in the RAM, and causes the display 204 to display the test list screen 241.

Though the description is made in FIG. 6 for a case where change of imaging site, imaging error and re-imaging occur as unexpected events, at least one of unexpected events of the following (a) to (i) is included as the unexpected event recorded by the controller 201.

(a) change of order contents of test order information which has been issued (for example, change, addition, cancelation of the imaging site)

(b) correction of patient information and/or attribute in the test order information

(c) imaging exceeding the imaging time which was determined in advance

(d) re-imaging

(e) imaging error

(f) image adjustment

(g) re-output of image

(h) screen shift to a screen other than the screen which was determined in advance

(i) adjustment work of test device

In such a way, in the test device 2, the console 21 records the test time of each test, the imaging time of each imaging in the test, the number of occurrence of the unexpected events, the working time of each unexpected event, and the like. Thus, it is possible to provide the occurrence situations of unexpected events in the test. In the embodiment, both of the number of occurrence of the unexpected events and the working time of each unexpected event are recorded. However, only one of them may be recorded.

### (Operation of Analysis Processing Device 3)

Next, the operation of the analysis processing device 3 will be described.

When the recording data is received from the order issuing device 1, the controller of the analysis processing device 3 saves the received recording data in the database of the storage so as to be associated with the device ID of the order issuing device 1 which is the transmission source.

The controller of the analysis processing device 3 performs analysis of the collected recording data for each predetermined period or according to user's operation, and calculates, for each day within the week and for each time zone, for example, the performance value (actual working value) of the test time measured by the order issuing device 1, the number of occurrence, the working times and the like of unexpected events for each operator. The period of the recording data used in analysis can be specified according to user's operation (for example, last one month, two months, one year, same month in the previous year, or the like). The controller generates the analysis result report and outputs the report to the terminal device 4 and the like.

When the recording data is received from the console 21, the controller of the analysis processing device 3 saves the received recording data in the database of the storage so as to be associated with the device ID of the console 21 of the transmission source. The data saving period of the recording data is console 21<analysis processing device 3. The controller of the analysis processing device 3 performs analysis of the collected recording data for each predetermined period or according to user's operation, and calculates the performance value, reference value thereof and the like for each imaging room, test device (panel), technician, imaging site, day of the week and time zone. The period of the recording data used in analysis can be specified according to user's operation (for example, last one month, two months, one year, same month in the previous year, or the like). The number of occurrence and the working time of the unexpected event are calculated for each item of the unexpected events, and a total for all the items is calculated.

For example, the controller of the analysis processing device 3 calculates the following (1) to (11). The following (1) to (7) are values (performance values) regarding performances of tests (imaging) for each imaging room, test device (panel), technician, imaging site, day of the week, and time zone.

(1) the number of tests, number of imaging, number of occurrence of unexpected events, and working time for each imaging room
(2) the number of tests, number of imaging, number of occurrence of unexpected events, and working time for each test device (for example, for each FPD)
(3) the number of tests, number of imaging, number of occurrence of unexpected events, and working time for each technician
(4) the number of tests, number of imaging, number of occurrence of unexpected events, and working time for each day of the week
(5) the number of tests, number of imaging, number of occurrence of unexpected events, and working time for each time zone
(6) imaging time for each technician
(7) imaging time for each site
(8) accumulated unexpected time (lost time, a total of working times of unexpected events during the period)
(9) ideal test time=actual test time−accumulated unexpected time
(10) reference value for each performance value of (1) to (7)

For example, the following value is calculated as the reference value.
As for (1) to (7), the representative value (for example, minimum value, average value, median value, or the like) of the performance value in the facility during the period
In a case of (3), the minimum value, average value or the median value of the performance value of a specific doctor in the facility during the period may be used.
Performance value in the past (for example, same month in the previous year, previous month)
   Alternatively, the manager may calculate (determine) the reference value on the basis of the experience.
   (11) difference value between the performance value and the reference value

As for (1) to (7), the representative value (average value, maximum value, minimum value, median value, or the like) for each day or during the period may be calculated as the performance value. The above (1) to (11) are examples, and only a part of them may be calculated, or other performance values or reference values may be calculated.

Next, the controller of the analysis processing device 3 generates the analysis result report on the basis of the calculated performance values and the like, and provides (outputs) the generated report according to the request from the terminal device 4 and the like.

In the analysis result report, for example, the above performance values and the reference values are displayed simultaneously. Alternatively, the difference values between the performance values and the reference values are displayed with graphs and numerical values as time series data.

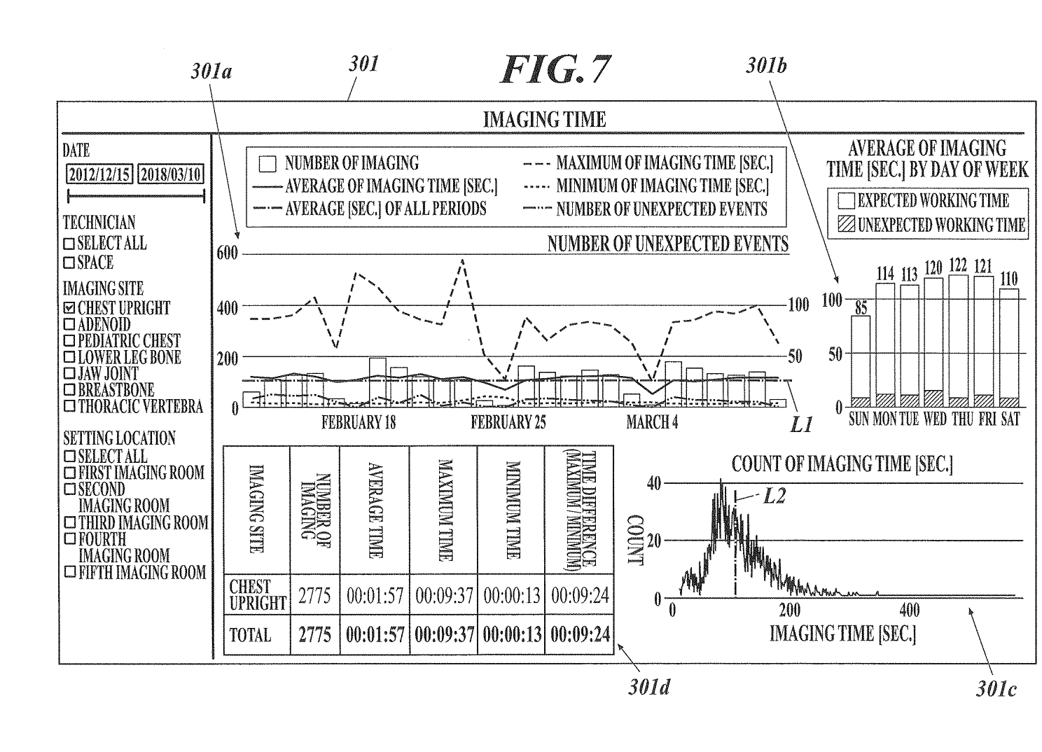

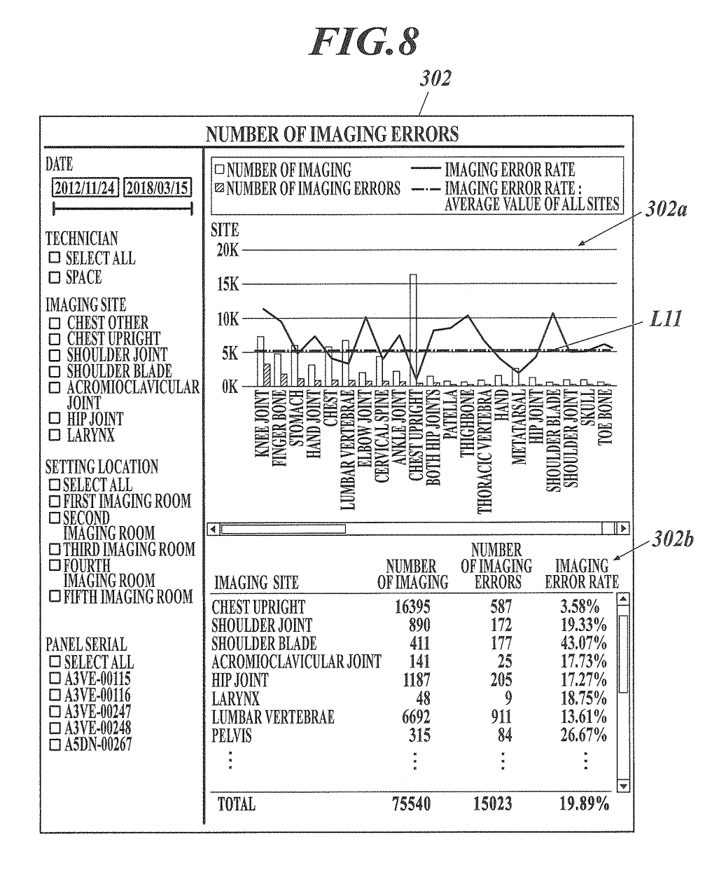

FIG. 7 and FIG. 8 are views showing examples of analysis result reports generated by the controller of the analysis processing device 3.

FIG. 7 shows, as an example, the analysis result report 301 regarding the imaging time for the chest upright. As shown in FIG. 7, there are displayed, in the analysis result report 301, a graph 301a showing the actual imaging time for each day during the predetermined period, a graph 301b showing the expected working time and the unexpected working time for each day of the week, a histogram 301c showing the count (number) for each imaging time during the period, and numerical values 301d of the number of imaging during the period, average imaging time, maximum imaging time, minimum imaging time, and the time difference between the maximum imaging time and the minimum imaging time.

In the graph 301a, for each day in the predetermined period, the number of imaging is displayed by a bar graph, and the average value, maximum value and the minimum value of the imaging time are displayed by line graphs. The position of the average value (reference value) of the imaging time in the facility during the predetermined period is indicated by the reference line L1. Moreover, for each day, the number of unexpected events is also displayed by a line graph. That is, in the graph 301a, the difference between the average (performance value) and the reference value of the imaging time for each day is displayed by a graph as time series data. Each value on the graphs and the numerical values of the differences may be displayed. The manager can easily recognize the day on which the average of the imaging time exceeds the reference value (day on which it took time to perform imaging) by viewing the graph 301a. The manager can attempt to make improvements by assuming that the generation of the unexpected events causes the increase of imaging time if the number of occurrence of the unexpected events is large on the day on which the average of the imaging times exceeds the reference value, for example.

In the graph 301b, the expected working time and the unexpected working time for each day of the week are shown by bar graphs. The manager can grasp the day of the week on which the unexpected working time is long by the graph 301b. For example, if the unexpected working time of a specific day of the week is long, the manager can attempt to specify the cause and make improvements.

In the histogram 301c, the reference line L2 is displayed at the position of the reference value. The manager can grasp the number of the imaging times which are longer than the reference and the number of imaging times which are shorter than the reference.

FIG. 8 shows the analysis result report 302 regarding the number of imaging errors for each imaging site as an example of the analysis result regarding the unexpected event. As shown in FIG. 8, in the analysis result report 302, there are displayed a graph 302a showing actual values of imaging errors for each imaging site during the predetermined period, and numerical values 302b showing actual values of imaging errors for each imaging site during the predetermined period.

In the graph 302a, for each imaging site, the number of imaging and the number of imaging errors are shown by bar graphs, and the imaging error rate is shown by a line graph. The position of the average (reference value) of the imaging error rates of all the sites is shown by the reference line L11. The manager can grasp how many imaging errors occurred with respect to the number of imaging for each imaging site during the predetermined period and which site has many imaging errors (which site has a problem in imaging), and attempt to make improvements of the imaging flow or the like for the imaging site generating many imaging errors.

In this way, in the analysis processing device 3, the performance value for each imaging room, each test device, each test technician and the like is calculated to generate the analysis result report on the basis of the recording data transmitted from the order issuing device 1 and the test device 2. Thus, the manager can easily grasp and manage the performance including the occurrence situations of unexpected events for each imaging room, each test device, each technician, each imaging site, and the like, and can consider the improvement plan.

The analysis result report may be provided as a Web content, provided as an electronic file (such as PDF), or may be provided via an e-mail. The analysis result report may be printed on a sheet.

The controller of the analysis processing device 3 causes the communicator to transmit recording data according to the request from the analysis processing device 6.

### (Operation of Terminal Device 4)

The terminal device 4 accesses the analysis processing device 3 and the analysis processing device 6 according to user's operation, and obtains and displays the analysis result report.

### (Operation of Analysis Processing Device 6)

The controller of the analysis processing device 6 obtains the recording data from the analysis processing device 3 of each of the medical facilities A to C for each predetermined period, and performs analysis integrating the recording data of all the facilities. For example, though the reference value calculated in the analysis processing device 3 was calculated with the recording data of the facility as a parameter, the controller calculates the performance value for each facility, imaging room, test device, test technician, imaging site, day of the week, and time zone, for example, with the recording data of all the facilities which are medical facilities A to C as a parameter. The controller generates the analysis report on the basis of the analysis result, and provides (outputs) the generated analysis report according to the request from the terminal device 4 or the like.

As described above, in the analysis processing system 100, the console 21 of the test device 2 records the test time from start of the test to end of the test for each test, the imaging time from start of imaging to end of the imaging for each imaging included in each test, and information regarding events which occurred in each test and deviate from the operation determined in advance, for example, the number of occurrence of the events and the working times.

Accordingly, it is possible to provide information on the occurrence situations of unexpected events in the test.

The event deviating from the operation determined in advance includes at least one of change of order contents of issued test order information, correction of patient information and/or patient attribute information, imaging exceeding the imaging time determined in advance, re-imaging, imaging error, image adjustment, re-output of image, shift to the screen other than the screen determined in advance, and adjustment work of the test device. Accordingly, it is possible to provide the occurrence situations of these events.

The analysis processing device 3 and the analysis processing device 6 analyze the recording data which was recorded by the console 21. For example, on the basis of the recording data, the performance value for each imaging room, test device, technician, and/or imaging site is calculated. Accordingly, it is possible to provide the performance value including the occurrence situations of unexpected events for each imaging room, test device, technician, and/or imaging site. Thus, the manager can easily grasp and manage the performance including the occurrence situations of unexpected events for each imaging room, test device, technician and/or imaging site, and consider the improvement plan.

The order issuing device 1 records the test time from start of the test to end of the test based on each piece of test order information which was issued, and information regarding the event deviating from the operation which was determined in advance. Accordingly, it is possible to provide the occurrence situations of unexpected events when the order of test is issued.

The analysis processing device 3 and the analysis processing device 6 analyze the data recorded in the test order information. Accordingly, it is possible to provide the performance value including the occurrence situations of unexpected events when the order of test was issued. Thus, the manager can easily grasp and manage the performance including the occurrence situations of unexpected events at the time of issuance of the order of test, and consider the improvement plan.

The described contents in the above embodiment are a preferred example of the present invention, and the present invention is not limited to the described contents.

For example, in the embodiment, as the recording data recorded by the console 21, there are recorded the test ID, test date and time, technician ID, imaging room ID, test time, panel ID, imaging date and time, imaging site and imaging time for each imaging, event ID, occurrence date and time, working time and the number of occurrence of the unexpected events which occurred in the test, and the like. However, the data to be recorded do not need to be all of them. It is sufficient that data necessary for the analysis to be performed is recorded.

In addition, for example, the above description discloses an example using a hard disk, a semiconductor non-volatile memory or the like as a computer readable medium of the program according to the present invention. However, the present invention is not limited to this example. A portable type recording medium such as a CD-ROM can be applied as other computer readable medium. A carrier wave is also applied as a medium providing the program data according to the present invention via the communication line.

As for the other detailed configurations and the detailed operations of devices forming the analysis processing system, modifications can be made as needed within the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-076492, filed on Apr. 12, 2018, including description, claims, drawings and abstract is incorporated herein by reference in its entirety.

What is claimed is:

1. A recording device which records information regarding a test that is performed based on test order information including imaging, the recording device comprising a hardware processor which records a test time of each test from start of the test to end of the test, an imaging time of each imaging included in each test from start of the imaging to end of the imaging, and information regarding an event which occurs in each test and deviates from an operation determined in advance.

2. The recording device according to claim 1, wherein the information regarding the event which deviates from the operation determined in advance includes at least a number of occurrence or a working time of the event.

3. The recording device according to claim 1, wherein the event which deviates from the operation determined in advance includes at least one of change of an order content of the issued test order information, correction of patient information and/or patient attribute information, imaging which exceeds an imaging time determined in advance, re-imaging, imaging error, image adjustment, image re-output, shift to a screen other than a screen which is determined in advance, and adjustment work of the test device.

4. An analysis processing system, comprising:
the recording device according to claim 1; and
an analysis processing device which analyzes data recorded by the recording device.

5. The analysis processing system according to claim 4, wherein
the recording device further records identification information of an imaging room in which each test is performed, a test device, a technician and/or an imaging site, and
the analysis processing device calculates a performance value for each imaging room, each test device, each technician and/or each imaging site from the data recorded by the recording device.

6. An order issuing device which issues test order information, the order issuing device comprising a hardware processor which records a test time and information regarding an event, the test time being a test time from start of a test to end of the test based on each piece of test order information that is issued, and the event deviating from an operation determined in advance.

7. An analysis processing system, comprising:
the order issuing device according to claim 6; and
an analysis processing device which analyzes data recorded by the order issuing device.
